(19) United States
(12) Patent Application Publication
(10) Pub. No.: US 2008/0194010 A1
(43) Pub. Date: Aug. 14, 2008

(54) Three Dimensional Cell Culture Construct and Apparatus for its Making

(75) Inventor: Qing Liu, Hillsborough, NJ (US)

Correspondence Address: PATENT DOCKET ADMINISTRATOR;LOWENSTEIN SANDLER PC, 65 LIVINGSTON AVENUE, ROSELAND, NJ, 07068, US

(73) Assignee: 3D BIOTEK, LLC, North Brunswick, NJ (US)

(21) Appl. No.: 12/030,615

(22) Filed: Feb. 13, 2008

Related U.S. Application Data
(60) Provisional application No. 60/889,580, filed on Feb. 13, 2007.

(51) Int. Cl. C12M 1/00

(52) U.S. Cl. 435/283.1

(57) ABSTRACT

The present invention relates to a three dimensional construct formed from non-biodegradable and non-cytotoxic polymers that provide an internal and external space for living cells to attach, proliferate and differentiate. The construct is composed of polymer struts and/or fibers which are joined together in a designed 3 dimensional pattern. The 3 dimensional cell culture construct (cell culture insert) is intended to be used together with cell/tissue culture plate, tissue culture flask, bioreactor and the like under normal cell culture conditions. The invention further provides methods of making the 3 dimensional cell culture construct. Finally, the invention provides kits comprising one or more 3 dimensional porous cell culture construct in a package together with other cell culture supplies, such as tissue culture plate and flasks.

The present application claims benefit of provisional applications: 60/889,580; the disclosure of which is hereby incorporated by reference.

### 1. FIELD OF THE INVENTION

The present invention relates to a porous three dimensional cell culture construct for living cells to attach, proliferate, and differentiate, wherein the construct is made from a non-biodegradable polymer material, preferably from polystyrene, polypropylene, polycarbonate, polyamide and polyvinyl chloride. The invention further provides methods for forming and making the construct, specifically involving the use of layer by layer assembly of prefabricated structures. The cell culture construct could be used in conventional cell culture vessels, such as cell culture dishes, cell culture plates, cell culture flasks, cell culture bags and bioreactors.

### 2. BACKGROUND OF THE INVENTION

While culturing cells in two dimensions (2D) is a convenient method for preparing, observing and studying cells and their interactions with pharmaceuticals, biological factors and biomaterials in vitro. It does not mimic the cell growth fashion in vivo. In real living body, cells are often growing in three dimensional (3D) and building three dimensional living tissue or organ. Emerging evidence showed that 3D cell culture systems in vitro can facilitate the understanding of structure-function relationship in normal and pathological tissue conditions. In order to study such functional and morphological interactions, some investigators have explored the use of three-dimensional gel substrates such as collagen gel [Douglas W H J, Moorman G W, and Teel R W, In Vitro, 1976; 12:373-381], gelatin, fibrin, agarose and alginate [Gruber H E, Fisher E C Jr, Desai B, Stasky A A, Hoelscher G, Hanley E N, Exp. Cell Res, 1997, 235:13-21; Gruber H E, Stasky A A, Hanley E N Jr, Matrix Biol, 1997; 16:285-288]. In these gel systems, cells were cultured within the gel matrix where they grow in 3 dimensional fashion. Recent studies have shown that human annulus disc cells cultured in 3 dimensional alginate or agarose gel systems showed different morphology, increased proteoglycan synthesis compared to monolayer grown cells, and formation of multi-celled colonies with extracellular matrix deposited around and between cells [Gruber H E, Fisher E C Jr, Desai B, Stasky A A, Hoelscher G, Hanley E N, Exp. Cell Res, 1997, 235:13-21; Gruber H E, Stasky A A, Hanley E N Jr, Matrix Biol, 1997; 16:285-288]. Further more, the human annulus disc cells cultured in 3 dimensional alginate gel systems showed the evidence of Type I and II collagen production which was not found in mono-layer cell culture [Gruber H E and Hanley E N, Jr, B M C Musculoskeletal Disorders, 2000; 1:1]. In vitro animal cell growth in 3D promotes normal epithelial polarity and differentiation [Roskelley C D, Bissell M J, Biochem Cell Biol, 1995; 73(7-8):391-7]. Cells move and divide more quickly and have a characteristically asymmetric shape compared with that of cells in living tissue [Cukierman E, Pankov R, Stevens D R, Yamada K M, Science, 2001; 294(5547):1708-12].

Three dimensional cell culture was also used to study the interactions between cell and growth factor as well as cell and drug. For example, three dimensional cell culture of cancer cells allows to explore many basic questions related to cancer biology, as receptors for tumor development growth factors are expressed in different ways in comparison to the standard 2 dimensional tissue culture plates [Wang F. Weaver V M, Petersen O W, Larabell C A, Dedhar S, Briand P, Lupu R, Bissell M J. Proc Natl Acad Sci USA, 1998; 95(25): 14821-6; Jacks T, Weinberg R A. Cell, 2002;111(7):923-5]. For breast cancer, 3 dimensional culture provides a model system for understanding the regulation of cancer cell proliferation and for evaluation of different anticancer drugs [Bissell M J, Rizki A, Mian I S, Curr Opin Cell Biolm, 2003;15(6):753-62; Padron J M, van der Wilt C L, Smid K, Smitskamp-Wilms E, Backus H H, Pizao P E, Giaccone C, Peters G J. Crit Rev Oncol Hematol, 2000;36(2-3): 141-57]. There is a substantial amount of evidence that cells growing in 3D culture are more resistant to cytotoxic agents than cells in monolayer or dispersed culture. Many studies have demonstrated an elevated level of drug resistance of spheroids culture compared with cells in monolayers [Hoffman R M. Cancer Cells 1991; 3(3):86-92]. Initially, investigators attributed drug resistance of spheroids to poor diffusion of the drugs to interior cells but now it has been proved that only 3 dimensional culture accounts for drug resistance rather than mere inaccessibility to nutrients [Lawler E M, Miller F R, Heppner G H, In Vitro, 1983; 19(8):600-10; Miller B E, Miller F R, Heppner G H, Cancer Res, 1985; 45(9):4200-5]. Further study confirmed that 3D culture is a better model for the cytotoxic evaluation of anticancer drugs in vitro [Harpreet K. Dhiman, Alok R Ray, Amulya K Panda, Biomaterials, 2005; 26 979-986].

Growing evidence show that three-dimensional (3D) environment also reveals fundamental mechanisms of cell function and that 3D culture systems in vitro can facilitate the understanding of structure-function relationship in normal and pathological conditions [Abbott A. Nature, 2003; 424(6951):870-2; Hutmacher D W. J Biomater Sci Polym Ed, 2001; 12:107-24; Schmeichel K L. Bissell M J. J Cell Sci, 2003; 116(Pt12):2377-88; Zahir N, Weaver V M, Curr Opin Genet Dev, 2004; 14:71-80; Martin I, Wendt D, Heberer M, Trends Biotechnol, 2004; 22:80-6]. It is now well accepted that bone and cartilage-derived cells behave differently in a 3 dimensional (3D) than in a two-dimensional (2D) environment and that the 3D culture systems in vitro are mimicking the in vivo situation more closely than the two-dimensional (2D) cultures [Kale S, Biermann S, Edwards C, Tarowski C, Morris M, Long M W, Nat Biotechnol, 2000;18:954-8; Ferrera D, Poggi S, Biassoni C, Dickson G R, Astigiano S, Barbieri O, Favre A, Franzi A T, Strangio A, Federici A, Manduca P, Bone, 2002;30:718-25; Tallheden T, Karlsson C, Brunner A, Van Der Lee J, Hagg R, Tommasini R, Lindahl A. Osteoarthritis Cartilage, 2004; 12:525-35;]. In a recent study, three human osteogenic cell lines and normal human osteogenic (HOST) cells were cultured in 3D inside a hydroxypropylmethylcellulose hydrogel matrix. It was demonstrated that osteosarcoma cells proliferate as clonogenic spheroids and that HOST colonies survive for at least 3 weeks. Mineralization assay and gene expression analysis of osteoblastic markers and cytokines indicate that all the cells cultured in 3D in this hydrogel matrix exhibited a more mature differentiation status than cells cultured in monolayer on plastic cell culture plates [Trojani C, Weiss P, Michiels J F, Vinatier C, Guicheux J, Daculsi G, Gaudray P, Carle G F, Rochet N., Biomaterials, 2005; 26(27):5509-17].

So far the evidence has shown clearly that culturing cells in a 3D environment will offer tremendous advantages over 2D culture environment. However, with current 3D gel systems, the cultured cells are embedded within a gel matrix which makes the exchange of the nutrients and metabolic products of the cultured cells problematic because of the diffusion limitation of gels. Also, unlike culturing cells in 2D cell culture plates, in which case cells can be easily detached from the culture plate using a trypsin solution and then isolated by centrifugation, cells cultured in 3D gel systems are difficult to recover or isolate because the cultured cells are embedded within the gel. In addition, culturing cells within a gel matrix requires preparation of the gel system each time before the culture, which is not only inconvenient to the researchers, especially when large quantities of cultures need to be prepared, but also introduces inconsistencies between the different batches of gel preparations due to slight variations in gel preparation among different researchers and laboratories.

Due to the above mentioned problems associated with the use of currently available 3D gel culture systems, 2D cell culture is still the preferred cell culture method despite the advantages that the 3D culture offers. Therefore, a 3D culture system which will offer all the convenience of a current 2D cell culture system will be extremely valuable to the pharmaceutical, life science and bioengineering research fields. The ideal 3D culture system will have the following characteristics:
1. It is a 3D structure that allows cellular adhesion on its external surface and inner space promoting 3D cellular or tissue formation.
2. It has struts or fibers aligned horizontally, vertically or obliquely that provides a surface or an inner lattice for cellular adhesion.
3. It has a porous 3D structure so the cells can attach to both the outer surface and inner surface of the 3D structure. The porous structure will allow for relative easy exchange of nutrients and metabolic products.
4. The 3D structure should be ready to use together with the current 2D cell culture plates and dishes as well as bioreactors. A 3D construct can simply be placed inside the wells of the 2D cell culture dishes or plates or the chamber of a bioreactor.
5. The structure should be made from non-cytotoxic and non-biodegradable materials, such as the materials used in current 2D cell culture system (polystyrene in particular). Non-cytotoxic does not mean that no cell dies or is not affected negatively, but that the general cell population is viable in the in vitro condition as provided.
6. The structure should be robust enough to withstand the normal mechanical handling of cell culture procedures without deforming and change the structure during the cell culture process.

Polystyrene, polyethylene, polyethylene terephthalate, polypropylene and polycarbonate are non-degradable polymer and have been used as substrate material for conducting two-dimensional (2D) cell culture. Cell culture vessels and membranes made from the above mentioned polymers are widely used and commercially available in many different sizes and configurations from many suppliers. Since these polymers are quite familiar to the researchers who are doing cell or tissue culture, it is conceivable that a 3D cell culture system made from these polymers would offer not only the advantages of a 3D culture environment, but also offer many other advantages that a 2D cell culture system could not offer, such as a well defined surface property and ease of use.

The use of polystyrene in fabrication of 3D matrix for cell culture has been scantily explored. Recently, Baker et al. (Baker et al., Biomaterials, 2006; 27, 3136-46) reported that they fabricated a 3D porous fibrous polystyrene matrix using an electro-spinning technique. The fibrous 3D polystyrene matrix obtained was a non-woven mat where the inter-fibrous space served as the porous space. The study suggested that these polystyrene 3D fibrous scaffolds complemented the 2D polystyrene cell culture plate systems. However, the disadvantages of these fibrous polystyrene matrixes are the following: the fiber size is difficult to control; the size of the pore and the shape of the matrix are not well defined; the average pore size was small (˜15 microns), and the fibrous matrix are soft in nature which makes it difficult for further cell culture manipulation without deforming the matrix. The average size of mammalian cells is between 10 to 100 microns.

Other researchers have also tried to make a more robust porous polystyrene matrix for routine cell culture. They used a high internal phase emulsion (HIPE) as a template to create the porous polystyrene structure (Hayman, et al, J. Biochemical and Biophysical Methods, 2005, 62:231-240). Highly porous polystyrene foams were prepared from poly(styrene/divinylbenzene) system. Studies have shown that human neurons adhered well to poly-d-lysine coated surfaces and extended neural processes. Neurite outgrowth was particularly enhanced when the surface also received a coating of laminin. However, there are also some disadvantages associated with the polystyrene foams, such as pore size and pore distribution cannot be very well controlled due to the inherent nature of this foaming process, the very tortuous, porous structure also makes the nutrient exchange difficult.

Due to above mentioned drawbacks associated with the use of current available 3D culture matrix, 2D cell culture is still the primary cell culture method despite the advantages that the 3D culture can offer. Therefore, a 3D culture system which has well defined pore size and porosity for routine 3 dimensional cell culture would be extremely valuable. The present invention provides methods to fabricate 3D cell culture construct which can be used as an insert to the cell culture vessels for conducting 3D cell culture.

### 3. SUMMARY OF THE INVENTION

### 3.1 CELL CULTURE CONSTRUCT

It is therefore an object of the present invention to provide a non-degradable porous 3D cell culture construct for use with current 2D tissue culture systems, such as tissue culture plates, for cell culture applications. It is also an object of the present invention to provide methods to fabricate 3D cell culture constructs that provide internal and external space for cellular adhesion. This cell culture construct has a well defined structure, including the porosity, pore size, surface area and surface chemistry. Preferably, the cell culture construct is made from a non-degradable polymer material. The polymer material is preferably polystyrene, which is being used in making 2D multi-well cell/tissue culture plate and flasks.

The surface area, porosity and pore size is determined by the design of the constructs, including the size and geometry of the struts, number of the struts/fibers in each unit volume and the construction pattern of the struts/fibers in the 3D construct structure. A strut is a structural component as is a fiber. A fiber is a discrete elongated piece, similar to lengths of a thread.

In one embodiment, therefore, the invention provides a 3D porous cell culture construct comprising struts and/or fibers joined in a rigid porous 3D pattern for cells to attach in cell culture medium. A construct is considered rigid when its final manifestation/composition after construction maintains or substantially maintains its shape under routine manipulation.

In one embodiment, therefore, the invention provides a 3D porous cell culture construct comprising struts and/or fibers joined in a porous 3D pattern for cells to attach in cell culture medium, the construct having an average pore size of between 15 microns and 1000 microns, between 25 microns and 500 microns, or between 50 microns and 100 microns.

In one embodiment, therefore, the invention provides a 3D porous cell culture construct comprising struts and/or fibers joined in a porous 3D pattern for cells to attach in cell culture medium, the construct having a pore distribution of greater than about 50%, greater than about 80% or greater thank about 95%.

In a specific embodiment, said cell culture construct is a 3 dimensional porous structure with evenly distributed pores at any given horizontal plane. Even distribution is determined by the number of pores at a given area compared with the number of pores at another given area where the area has the same size and dimension. Absolute even distribution of pores for a plane comparing two equivalent areas of the plane is 100%. A plane is considered evenly distributed if the ratio of the number of pores between two equivalent areas (comparing area with the lesser pore number to the area with the greater pore number) of the same plane is greater than 50%, or greater than 80%, or greater than 95%.

In one embodiment, therefore, the invention provides a 3D cell culture construct composed of struts and/or sturdy fibers which are jointed together in an angle at the joint (FIGS. 2 and 4). Further, the struts and/or fibers can be woven together.

In one embodiment, therefore, the invention provides a 3D cell culture construct composed of struts and/or sturdy fibers which are perpendiculars to each other at the joint (FIGS. 1-3, 5). In other embodiments, the struts and/or fibers are joined at an acute angle (less than 90°) or at an obtuse angle (more than 90°).

In a specific embodiment, said cell culture construct is a 3 dimensional disc shaped porous structure. In another specific embodiment, the cell culture construct is a 3 dimensional cubical shaped porous structure.

In one embodiment, therefore, the invention provides a 3D cell culture construct composed of struts and/or sturdy fibers are polymers. The construct can have struts and/or fibers of constant diameter or variable diameters with cross sections of the struts and/or fibers being various shapes.

In one embodiment, therefore, the invention provides a 3D cell culture construct composed of struts and/or sturdy fibers which are positioned horizontally, vertically or obliquely relative to a base providing a space for cells to intercalate and form 3D adhesion with each other and with the struts and fibers.

In one embodiment, therefore, the invention provides a 3D porous cell culture construct comprising struts and/or fibers joined in a porous 3D pattern for cells to attach in cell culture medium, the construct having pores of constant size and/or dimension or pores of variable size and/or dimension.

In one embodiment, therefore, the invention provides a 3D cell culture construct composed of struts and/or fibers and the struts and fibers are joined together in a pre-designed fashion or pattern. In a specific embodiment, said cell culture construct is composed of struts and fibers made from non-cytotoxic and non-degradable polymers. Materials are considered non-cytotoxic and non-degradable as standard materials currently used for cell culture purposes (e.g., cell culture plates and dishes). In a more specific embodiment, said non-degradable polymer is polystyrene.

In a specific embodiment, the cell culture construct is impregnated with one or more biomolecules. A biomolecule can be a protein, peptide, glycoaminoglycan, a naturally occurring compound or polymer, a therapeutic agent or a combination thereof.

Another method of growing cells on three dimensional cell culture construct is immersing a cell culture construct in a cell suspension within a spinner flask and the flask is placed in an incubator appropriate for cellular maintenance. The cells in cellular suspension are then allowed a sufficient period of time to attach to the cell culture construct, and followed by submerging the cell culture construct in a growth medium inside a cell culture apparatus such as a cell culture plate, dish or bioreactor.

In a specific embodiment, the invention is a method of making a cell culture insert. A cell culture insert is assembled by adding successive layers comprising struts and/or fibers. The surface of the assembled cell culture insert is treated by plasma treatment or surface coating. Finally, the cell culture insert is sterilized using radiation and packaged. Further, the polymer processing method used is injection molding, fiber weaving, bonding or a combination thereof.

In a specific embodiment, the invention is a method of making a three dimensional porous cell culture construct. A cell culture insert is assembled by adding successive layers comprising struts and/or fibers and by altering the number of the struts and/or fibers for a given volume of the cell culture construct or by altering the diameter of the struts and/or fibers for a given volume of the cell culture construct.

In a specific embodiment, the invention is a method of making a three dimensional porous cell culture construct. A cell culture insert is assembled by adding successive layers comprising struts and/or fibers and positioning the struts and/or fibers relative to each other at an angle to provide a pore of predetermined size and dimension. The angle of the struts and/or fibers to each other is about perpendicular (90°). Alternatively, the angle could be acute (less than 90°) or obtuse (greater 90°).

In a specific embodiment, the invention is a kit of a three dimensional porous cell culture construct.

### 4. BRIEF DESCRIPTION OF THE FIGURES

FIG. 1. This figure depicts a cell culture construct, comprising multi layers aligned polymer fibers joined and assembled together.

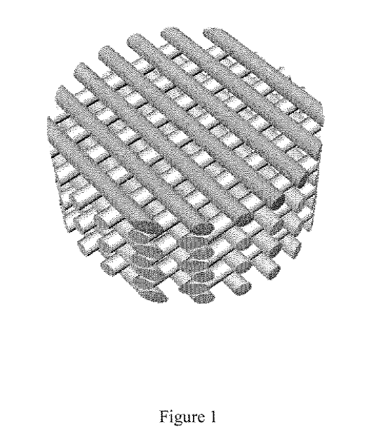

FIG. 2. This figure depicts a cross section of one embodiment of the cell culture construct showing the fiber orientation and the way that the fibers are Joined together.

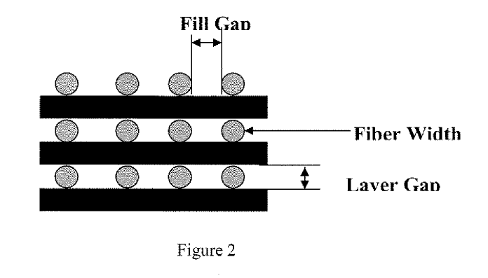

FIG. 3. This figure depicts an assembled, prefabricated layers to produce the embodiment in FIG. 1.

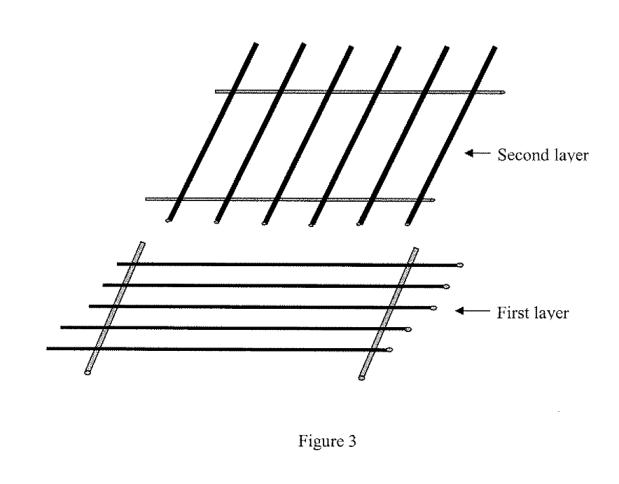

FIG. 4. This figure depicts another embodiment of a cell culture construct, comprising multi layers aligned polymer fibers joined and assembled together.

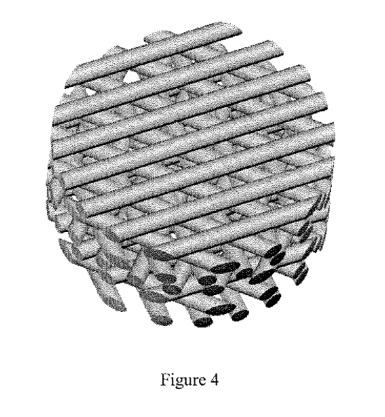

FIG. 5. This figure depicts another embodiment of assembled prefabricated layers that produce a different configuration of the 3D cell culture construct.

FIG. 6. This figure depicts another embodiment of assembled prefabricated layers that produce a different configuration of the 3D cell culture construct.

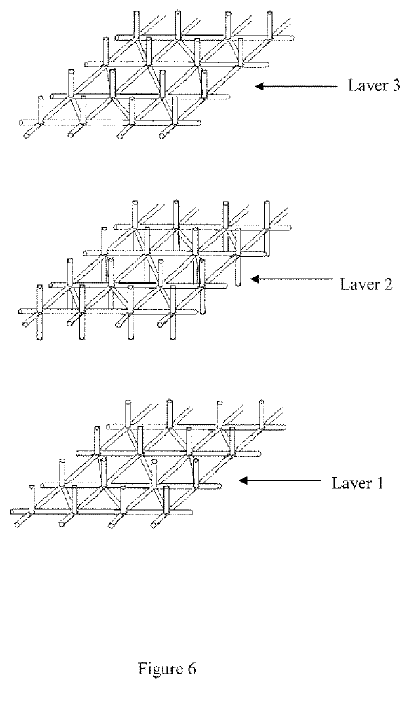

FIG. 7. This figure depicts fiber clap used to assemble the prefabricated layers and secure them in position in the 3D cell culture construct.

### 5. DETAILED DESCRIPTION OF THE INVENTION

The present invention provides a 3 dimensional cell culture construct that provides an internal and external space for cellular adhesion made from a non-degradable polymer material, preferably a polystyrene or another polymer which has been used to fabricate tissue culture plates and flasks. The cell culture construct is composed of multi-layers of interconnected struts and/or sturdy fibers which are joined together in a pre-design fashion or pattern. Such a configuration allows the cell culture construct to have 100% pore interconnection. In addition to the cell culture construct, the present invention also provides methods of making the cell culture construct, and of using the cell culture construct in a cell culture research setting.

### 5.1 CONFIGURATIONS

The cell culture construct of the present invention may be configured in any size and shape to accomplish the particular purpose at hand, e.g., size and shape which fits into cell/tissue culture plate, flasks, and bioreactors.

In one embodiment, therefore, the invention provides a 3D cell culture construct composed of struts and/or fibers. The struts and fibers are joined together in a pre-designed fashion or pattern. In one embodiment, the struts and/or fibers are joined at a perpendicular angle. In other embodiments, the struts and/or fibers are joined at an acute angle (less than 90°) or at an obtuse angle (more than 90°).

The surface area, porosity and pore size of the cell culture construct is determined by the design of the constructs, including the size and geometry of the struts, number of the struts in each unit volume and the construction pattern of the struts in the 3D construct structure.

In one embodiment, therefore, the invention provides a 3D porous cell culture construct comprising struts and/or fibers joined in a porous 3D pattern for cells to attach in cell culture medium, the construct having struts and/or fibers with a constant diameter or having struts and/or fibers with different diameters. In addition, the cross sections of the struts and/or fibers could be a circle, triangle, square, rectangle, star, or irregular shape.

In one embodiment, therefore, the invention provides a 3D cell culture construct composed of struts and/or sturdy fibers which are positioned horizontally, vertically or obliquely relative to a base providing a space for cells to intercalate and form 3D adhesion with each other and with the struts and fibers.

In one embodiment, therefore, the invention provides a 3D cell culture construct composed of struts and/or sturdy fibers which are perpendiculars to each other at the joints.

In one embodiment, therefore, the invention provides a 3D cell culture construct composed of struts and/or fibers which are not all perpendiculars to each other at the joint but are jointed at different angles.

In a specific embodiment, said cell culture construct is a 3 dimensional disc shaped porous structure. In another specific embodiment, said cell culture construct is a cubical 3 dimensional shaped porous structure.

In a specific embodiment, said cell culture construct has pores of constant size and/or dimension or pores of variable size and/or dimension. In addition, the construct can have pores of constant size and/or dimension for each plane, but the pores are on each plane differ from plane to plane in terms of size and/or dimension. Alternatively, the change in pore size and/or dimension can just be one or a few pores on a plane relative to pores on other planes. Further, the size and/or dimension for the pores on each plane could decrease or increase in size.

### 5.1.1 Dimensions

The cell culture construct of the invention may be pre-fabricated to standard sizes, or may be custom-made to fit into a particular cell culture plate well, chamber, flask, bioreactor. In one embodiment, therefore, the invention provides a cell culture construct with a size (both diameter and height) that fits into a round well of a tissue culture plate that are commercially available. In another embodiment, the invention provides a cell culture construct with a cubic shape size (length×width×height) that fits into a rectangular well of a tissue culture plate. In another embodiment, the cell culture construct has a size and shape that fits into chamber of a bioreactor. In a specific embodiments, the size of the cell culture construct fits into a tissue culture flask.

The diameter of the struts/fibers of the 3D cell culture construct may vary from 50 nm to 1 mm.

The mean pore size of the cell culture constructs may vary from 50 nm to 1 mm.

### 5.2 MATERIALS

The cell culture construct of the present invention is made primarily, or exclusively, of a non-degradable polymer. Such non-degradable polymers include, for example, non-degradable synthetic polymers such as, but are not limited to, polystyrene, polyethylene, polypropylene, polycarbonate, polyethylene terephthalate, polyamide, polyvinyle chloride etc.

The cell culture construct can be impregnated with one or more biomolecules. A biomolecule can be a protein, peptide, glycoaminoglycan, a naturally occurring compound or polymer, a therapeutic agent or a combination thereof Examples of naturally occurring compound or polymer are collagen, laminin, or fibronectin. Therapeutic agents include but are not limited to, antibiotics, hormones, growth factors, anti-tumor agents, anti-fungal agents, anti-viral agents, pain medications, anti-histamines, anti-inflammatory agents, anti-infective, wound healing agents, wound sealants, cellular attractants, cytokines and the like. A therapeutic agent is anything that when applied to cell would benefit human health.

Antibiotics are chemotherapeutic agents that inhibit or abolish the growth of micro-organisms, such as bacteria, fungi, or protozoans. Examples of common antibiotics are penicillin and streptomycin. Other known antibiotics are amikacin, gentamicin, kanamycin, neomycin, netilmicin, tobramycin, paromomycin, geldanamycin, herbimycin, loracarbef, ertapenem, doripenem, imipenem/cilastatin, meropenem, cefadroxil, cefazolin, cefalotin or cefalothin, cefalexin, cefaclor, cefamandole, cefoxitin, cefprozil, cefuroxime, cefixime, cefdinir, cefditorern, cefoperazone, cefotaxime, cefpodoxime, ceftazidime, ceftibuten, ceftizoxime, ceftriaxone, cedinir, cefepime, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, nafcillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, prontosil, sulfacetamide, slfamethizole, slfanilimide, sulfasalazine, sulfisoxazole, trimethoprim, trimethoprim-sulfamethoxazole, demeclocycline, doxycycline, minocycline, oxvtetracycline, tetracycline, arsphenamine, chloramphenicol, clindamycin, lincoamycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin or rifampicin and tinidazole.

A hormone is a chemical messenger that carries a signal from one cell (or group of cells) to another via the blood. Examples of hormones are melatonin, serotonin, thyroxine, triiodothyronine, epinephrine, norepinephrine, dopamine, antimullerian hormone, adiponectin, adrenocorticotropic hormone, angiotensinogen and angiotensin, antidiuretic hormone, atrial-natriuretic peptide, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, insulin-like growth factor, leptin, luteinizing hormone, melanocyte stimulating hormone, oxytocin, parathyroid hormone, prolactin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide Y, histamine, endothelin, pancreatic polypeptide, renin, and enkephalin.

Growth factor refers to a naturally occurring protein capable of stimulating cellular proliferation and cellular differentiation. Examples are transforming growth factor beta (TGF-β), granulocyte-colony stimulating factor (G-CSF), granulocyte-macrophage colony stimulating factor (GM-CSF), nerve growth factor (NGF), neurotrophins, platelet-derived growth factor (PDGF), erythropoietin (EPO), thrombopoictin (TPO), myostatin (GDF-8), growth differentiation factor-9 (GDF9), acidic fibroblast growth factor (aFGF or FGF-1), basic fibroblast growth factor (bFGF or FGF-2), epidermal growth factor (EGF), and hepatocyte growth factor (HGF).

Antitumors or antineoplastics are drugs that inhibit and combat the development of tumors. Examples are actinomycin (e.g., actinomycin-D), anthracyclines (e.g. doxorubicin, daunorubicin, epirubicin), bleomycin, plicamycin, and mitomycin.

An anti-fungal agent is medication used to treat fungal infections. Examples are natamycin, rimocidin, filipin, nystatin, amphotericin B, miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, terbinafine, amorolfine, naftifine, butenafine, anidulafungin, caspofungin, micafungin, benzoic acid, ciclopirox, flucytosine, griseofulvin, gentian violet, haloprogin, tolnaftate, undecylenic acid, tea tree oil, citronella oil, lemon grass, orange oil, palmarosa oil, patchouli, lemon myrtle, neem seed oil, coconut oil, zinc, and selenium.

Antiviral agents are a class of medication used specifically for treating viral infections. Examples are abacavir aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors (fusion inhibitor), famcielovir, fomivirsen, fosamprenavir, foscarnet, fosfonet, gancielovir, gardasil, ibacitabine, imunovir, idoxuridine, imiquimod, indinavir, inosine, integrase inhibitor, interferon type III, interferon type II, interferon type I, lamivudine, lopinavir, loviride, MK-0518 (raltegravir), maraviroc, moroxydine, nelfinavir, nevirapine, nexavir, nucleoside analogues, oseltamivir, penciclovir, peramivir, pleconaril, podophyllotoxin, protease inhibitor (pharmacology), reverse transcriptase inhibitor, ribavirin, rimantadine, ritonavir, saquinavir, stavudine, synergistic enhancer (antiretroviral), tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

Pain medications or analgesics (colloquially known as a painkiller) are members of the diverse group of drugs used to relieve pain. Examples are paracetamol/acetaminophen, nonsteroidal anti-inflammatory drugs (NSAIDs), COX-2 inhibitors (e.g., r ofecoxib and celecoxib), morphine, codeine, oxycodone, hydrocodone, diamorphine, pethidine, tramadol, buprenorphine, tricyclic antidepressants (e.g., amitriptyline), carbamazepine, gabapentin and pregabalin.

An antihistamine is a histamine antagonist that serves to reduce or eliminate effects mediated by histamine, an endogenous chemical mediator released during allergic reactions. Examples are H1 antihistamine, aceprometazine, alimemazine, astemizole, azatadine, azelastine, benadryl, brompheniramine, chlorcyclizine, chloropyramine, chlorphenamine, phenylpropanolamine, cinnarizine, clemastine, cyclizine, cyproheptadine, dexbrompheniramine, dexchlorpheniramine, diphenhydramine, doxylamine, ebastine, emedastine, epinastine, fexofenadine, histamine antagonist (e.g., cimetidine, ranitidine, and famotidine; ABT-239, thioperamide, clobenpropit, impromidine, thioperamide, cromoglicate, nedocromil), hydroxyzine, ketotifen, levocabastine, mebhydrolin, mepyramine, mthapyrilene, methdilazine, olopatadine, pheniramine, phenyltoloxamine, resporal, semprex-D, sominex, talastine, terfenadine, and triprolidine.

Anti-inflammatory agent refers to a substance that reduces inflammation. Examples are corticosteroids, ibuprofen, diclofenac and naproxen, helenalin, salicylic acid, capsaicin, and omega-3 fatty acids.

Anti-infective agent is any agent capable of preventing or counteracting infection. It could be divided into several groups. Anthelminthics is one group of anti-infective agents comprising of albendazole, levamisole, mebendazole, niclosamide, praziquantel, and pyrantel. Another group is antifilarials, such as diethylcarbamazine, ivermectin, suramin sodium, antischistosomals and antitrematode medicine, oxamniquine, praziquantel, and triclabendazole. Another group is the antibacterials, which can be further subdivided. The beta lactam medicines are amoxicillin, ampicillin, benzathine benzylpenicillin, benzylpenicillin, cefazolin, cefixime, ceftazidime, ceftriaxone, cloxacillin, co-amoxiclav, imipenem/cilastatin, phenoxymethylpenicillin, and procaine benzylpenicillin. Other antibacterials are azithromycin, chloramphenicol, ciprofloxacin, clindamycin, co-trimoxazole, doxycycline, erythromycin, gentamicin, metronidazole, nitrofurantoin, spectinomycin, sulfadiazine, trimethoprim, and vancomycin. Examples of antileprosy medicines are clofazimine, dapsone, and rifampicin. Examples of antituberculosis medicines are amikacin, p-aminosalicylic acid, capreomycin, cycloserine, ethambutol, ethionamide, isoniazid, kanamycin, ofloxacin, pyrazinamide, rifampicin, and streptomycin. Examples of antifungal medicines are amphotericin B, clotrimazole, fluconazole, flucytosine, griseofulvin, nnystatin, potassium iodide. Antiviral agents are also anti-infective agents. An example of a antiherpes medicine is acyclovir. Examples of antiretrovirals are nucleoside/nucleotide reverse transcriptase inhibitors. Other examples are abacavir, didanosine, emtricitabine, lamivudine, stavudine, tenofovir disoproxil fumarate, zidovudine, non-nucleoside reverse transcriptase inhibitors, efavirenz, nevirapine, protease inhibitors, indinavir, lopinavir+ritonavir, nelfinavir, ritonavir, saquinavir and ribavirin. Examples of antiprotozoal medicines are antiamoebic and antigiardiasis medicines such as diloxanide, metronidazole; antileishmaniasis medicines such as amphotericin B, meglumine antimoniate, pentamidine; antimalarial medicines, such as amodiaquine, artemether, artemether+lumefantrine, artesunate, chloroquine, doxycycline, mefloquine, primaquine, quinine, sulfadoxine+pyrimethamine, chloroquine, and proguanil. Antipneumocytosis and antioxoplasmosis medicines are pentamindine, pyrimethamine, sulfamethoxazole+trimethoprim. Antitrypanosomal medicines are eflomithine, melarsoprol, pentamidine, suramin sodium, benznidazole, and nifitimox. Antimigraine medicines, acetylsalicylic acid, paracetamol, and propranolol.

Wound healing agents facilitate the body's natural process of regenerating dermal and epidermal tissue. Examples are fibrin, fibronectin, collagen, serotonin, bradykinin, prostaglandins, prostacyclins, thromboxane, histamine, neuropeptides, kinins, collagenases, plasminogen activator, zinc-dependent metalloproteinases, lactic acid, glycosaminoglycans, proteoglycans, glycoproteins, glycosaminoglycans (GAGs), elastin, growth factors (PDGF, TGF-β), nitric oxide, and matrix metalloproteinases, Examples of wound sealants are platelet gel and fibrin.

Cellular attractants or chemotaxic agents are chemicals or molecules in the environment that are sensed by bodily cells, bacteria, and other single-cell or multicellular organisms affecting their movements. Examples are amino acids, formyl peptides [e.g., N-formylmethionyl-leucyl-phenylalanine (fMLF or fMLP in references], complement 3a (C3a) and complement 5a (C5a), chemokines (e.g., IL-8); leukotrienes [e.g., leukotriene B4 (LTB4)].

Cytokines are group of proteins and peptides that are signalling compounds produced by animal cells to communicate with one another. Cytokines can be divided into several families. Examples are the four alpha-helix bundle family with three subfamilies: the IL-2 subfamily [e.g., erythropoietin (EPO) and thrombopoictin (THPO)], the interferon (IFN) subfamily, the IL-10 subfamily. Other examples are the IL-1 family (e.g., IL-1 and IL-18), the IL-17 family, chemokines, immunoglobulin (Ig) superfamily, haemopoietic growth factor (type 1) family, Interferon (type 2) family, tumor necrosis factors (TNF) (type 3) family, seven transmembrane helix family, and transforming growth factor beta superfamily.

The surface or partial surface of the cell culture construct can be further treated by a physiochemical mean, a chemical mean, a coating mean, or a combination thereof to improve cellular attachment.

The surface of the cell culture construct can be further treated with surface modification techniques pertaining to physiochemical means known in the art, such as, but not limited to, plasma or glow discharge, to improve the surface property of the construct for better cellular attachment.

The surface of the cell culture construct can be further surface treated by chemical means, particularly with acids or bases. In a specific embodiment, the cell culture construct is treated with H₂SO₄, HNO₃, HCl, H₃PO₄, H₂CrO₄, or a combination thereof. In a specific embodiment, the cell culture construct is treated with NaOH, KOH, Ba(OH)₂, CsOH, Sr(OH)₂Ca(OH)₂, LiOH, RbOH, or a combination thereof.

The surface of the cell culture construct can be further surface treated by coating means, which is applying a substance on the surface that is different from the material of the struts and/or fibers. The substance can be covalently bonded or physically absorbed to the surface of the struts and/or fibers. Alternatively, the substance can be bonded to the surface of the construct through hydrogen bonding, ionic bonding, Van der Waals force or a combination thereof. To increase the stability of the biological molecular coating, the coating can be crosslinked using various crosslinking technologies, such as chemical crosslinking, radiation, thermal treatment, or a combination thereof, etc. Further, the crosslinking can take place in a vacuum at an elevated temperature above room temperature. The radiation used for crosslinking can be e-beam radiation, gamma radiation, ultraviolet radiation, or a combination thereof.

The coating substance can be a protein, peptide, glycoaminoglycan, a naturally occurring substance, an inorganic substance, a therapeutic agent, or a combination thereof.

The surface of the cell culture construct can be further coated with biological molecules or naturally occurring compound or polymer, such as, but not limited to, collagen (type I, II, III, IV, V, IV, etc), fibronectin, laminin, or other extracellular matrix molecules. Examples of extracellular matrix molecules are heparan sulfate, chondroitin sulfate, keratan sulfates, hyaluronic acid, elastin, hemicellulose, pectin, and extensin. The biological molecules are either covalently bonded to the surface, or physically absorbed to the surface of the cell culture constructs.

The surface of the cell culture construct can be further surface coated with a synthetic polymer, such as, but not limited to, polyvinyl alcohol, polyethylene glycol, polyvinyl polypyrrolidone, poly(L-lactide), polylysine, etc.

The three dimensional porous cell culture construct can be coated with organic substance, such as gelatin, chitosan, polyacrylic acid, polyethylene glycol, polyvinyl alcohol, polyvinylpyrrolidone and a combination thereof.

In a specific embodiment, the cell culture construct is coated with an inorganic material, such as calcium phosphate, TiO₂, Al₂O₃, or a combination there of etc.

In a specific embodiment, the cell culture construct is coated with a composite coating of two or more organic materials, such as, but not limited to, gelatin and chitosan, polyacrylic acid and polyethylene glycol, polyvinyl alcohol and polyvinylpyrrolidone, etc.

In a specific embodiment, the cell culture construct is coated with a composite of inorganic materials, such as calcium phosphate and TiO₂, calcium phosphate and Al₂O₃, etc. The inorganic composite coating is either chemically bonded to the surface, or physically absorbed to the surface of the said cell culture constructs.

In a specific embodiment, the cell culture construct is coated with a composite coating of inorganic and organic materials, such as but not limited to, calcium phosphate/collagen, calcium phosphate/gelatin, calcium phosphate/polyethylene glycol, etc. The composite coating is either chemically bonded to the surface, or physically absorbed to the surface of the said cell culture constructs.

### 5.2.1.1 Method of Making Cell Culture Construct

The cell culture construct can be fabricated using several methods, such as, but not limited to, layer by layer assembling technique and layer by layer fabrication technique. Below is one example.

This method is what we describe as a scaffold assembling technique. One exemplary method for fabrication of the invented cell culture construct comprises the following steps.

Step I. Each layer of scaffold is pre-fabricated by a suitable polymer processing techniques according to the structure design. The polymer processing technique can be, but not limited to, injection molding and fiber woven process and bonding, which are the most efficient and cost effective ways to fabricate polymer parts and polymer screens.

Step II. The layers of the scaffold are then assembled together by putting several layers of the scaffolds on top of each other. Each layer of scaffolds may have different structure and may also be bigger in area than the area of a final product. When the area of the construct is bigger than the final desired product, the final desired product can be cut into the right size and shape from the assembled big construct using a mechanical device, such as a die cutter, or a laser beam. One or more final cell culture constructs may be cut from a single assembled big construct. Another embodiment is when each layer of the scaffold is prefabricated to the desired size; the cell culture construct is then assembled together with the aid of a mechanical device to guide and orient layers of the scaffold during the assembling process. For example, when making a disc shape cell culture construct, the mechanical device is a hollow tube having a right diameter which would accommodate several prefabricated circular scaffold parts. The tube guide may also have a mechanical mechanism that will align the prefabricated parts in certain way to achieve the predefined configuration after assembling. After scaffold parts are put together in position with the aid of a mechanical assembling device, the parts are then tied together using polymer fibers or clips, etc, which are non-cytotoxic and preferably are made from the same type of materials as that of cell culture construct. The same assembling process can be applied to assemble the cubic shape cell culture construct, using a mechanical assemble guide which having a square or rectangular cross section area.

The assemble guide can also be pre-aligned polymer fibers. These pre-aligned fibers will pass through some of the holes or pores of the prefabricated scaffold parts and finally tie these parts together to achieve predefined configuration after assembling.

This technique of scaffold assembling also provides the possibility to assemble a non uniform structure cell culture construct by putting together several pre-fabricated parts having several different structural designs. The cell culture construct structure can also be altered by changing the relative position of the one part to the others, e.g. by rotating some parts to a certain degree.

The benefit of fabricating cell culture constructs using the assembling technique described above is that the construct can be easily disassembled by simply removing the assembling clip or assembling fibers that hold the individual parts together after being used in cell culture. The disassembled parts can be easily evaluated by conventional microscopic techniques, such as light microscopy, scanning electron microscopy, etc.

The assembled cell culture construct can be further treated by various surface modification techniques, such as plasma and glow discharge techniques known to one skilled in the art. The cell culture construct can also be coated with inorganic, organic and inorganic/organic materials by dip coating, chemical grafting, and/or other techniques known to one skilled in the art. The surface treated cell culture construct can be packaged and sterilized.

### 5.3 KITS

The invention further comprises kits providing one or more of the cell culture constructs with tissue culture plate in one package container. Kits of the invention comprise one or more cell culture constructs, and may comprise other components, such as a mechanical device for taking out and inserting the cell culture construct into the tissue culture plate, sterile packaging foam or other disposables, and the like.

A kit of the invention may comprise a single cell culture construct, sterilely wrapped and ready for immediate use. In one embodiment, a kit of the invention may comprise two or more cell culture constructs of the same size. In another embodiment, the kit of the invention may comprise two or more cell culture constructs of the different sizes.

A kit of the invention may comprise a single cell culture construct or multiple cell culture constructs, which are inserted into the wells of a single or multiple cell/tissue culture plates, sterilely wrapped and ready for immediate use.

### 5.4 CELL CULTURE USE OF THE CELL CULTURE CONSTRUCT

### 5.4.1 Use with a Tissue Culture Polystyrene Plate

The present invention also provides methods of using the cell culture construct for culturing living cells within a tissue culture polystyrene plate. The cell culture construct can be a disc or cubic shape that fits into the well of a tissue culture plate. Cells can be seeded into the cell culture constructs using a dynamic seeding or static seeding method.

In one example using a static seeding method, a certain volume of cell suspension was piped onto the upper surface of the cell culture construct and allowed to attach for certain time before flooding with medium. After being seeded with cells, cell culture constructs were maintained in the well plates submerged in growth medium, and cultured at 37° C. in an incubator in a 90% humidified atmosphere of 5-10% carbon dioxide in air.

In another example using a dynamic seeding method, seeding was performed by immersing cell culture constructs in cell suspension within a spinner flask, and contained at 37° C. in a humidified 5% CO₂ incubator. After seeding, cell culture constructs were placed into wells of tissue culture plate with medium for further culture at 37° C. in a humidified 5% Co₂ incubator. Culture medium was replaced regularly.

After cell culture was finished at certain time point, the cell culture constructs were taken out of the cell culture plate and underwent conventional assays. Cell culture constructs were disassembled in order to visualize, under a microscope, the cellular attachment and cellular activities within different layers or locations of the cell culture constructs.

In the case where the cells need to be recovered, the cells were trypsinized using Trypsin-EDTA solution. After detaching from cell culture constructs, cells were re- suspend in a small volume of fresh serum-containing medium to inactivate the trypsin. These cells then could be used for other purposes.

### 5.4.2 Use with a Bioreactor

The present invention also provides methods of using the cell culture construct for culturing living cells within a bioreactor. The cell culture construct can be a disc or cubic shape and fits into the bioreactor.

In a example of using a static seeding method, a certain volume of cell suspension was pipetted onto the upper surface of the cell culture construct and allowed to attach for certain period of time before flooding with medium. After being seeded with cells with either static seeding or dynamic seeding method, these cell seeded cell culture constructs were maintained in a bioreactor submerged in growth medium, and cultured at 37° C. in a 90% humidified atmosphere of 5-10% carbon dioxide in air. Culture medium was replaced regularly and constantly circulated through the cell culture constructs.

After cell culture was finished at certain time point, the cell culture constructs were taken out of the cell culture plate and underwent conventional assays. Cell culture constructs were disassembled in order to visualize, under a microscope, the cellular attachment and cellular activities within different layers or locations of the cell culture constructs.

In the case where the cells need to be recovered, the cells were trypsinized using Trypsin-EDTA solution. After detaching from cell culture constructs, cells were re- suspend in a small volume of fresh serum-containing medium to inactivate the trypsin. These cells then could be used for other purposes.

### 5.5 EXAMPLE 1

### Method of Making Cell Culture Construct

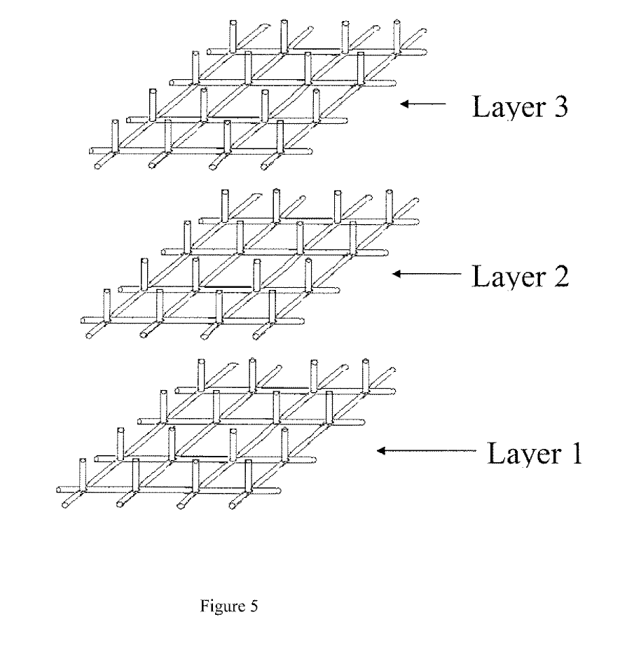

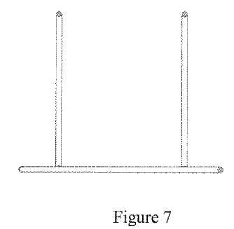

A cell culture construct is fabricated using polystyrene material. The cell culture construct parts as shown in FIG. 5 were used to assemble into cell culture construct. These parts were injection molded according to the design. After the parts were made, the first layer was placed first in the assembling guide and then followed by sequentially putting the second, third and forth layers of part into the guide. So the total number of the parts was 4. These 4 parts were then tied together using a polystyrene fiber clap as shown in FIG. 7. The two ends of the clap were further secured by forming a tie or deforming the two ends so that the two ends would not coming out through the holes of the construct. After assembled, the cell culture construct was plasma-treated in argon using a Polaron PT7300 RF Plasma Barrel Etcher (Quorum Technology, East Sussex, UK). The radio-frequency power, pressure and treatment time were fixed at 296 W, at 1×10⁻¹ mbar and 5 min, respectively.

The plasma treated cell culture construct was individually packaged and terminally sterilized using y ray radiation at a dose of 20 KGy.

### 5.6 EXAMPLE 2

### Use of Cell Culture Construct for Cell Culture

The present invention also provides methods of using the cell culture construct for culturing living cells within a tissue culture polystyrene plate. The cell culture construct used in this study had a size of 10 mm wide×10 mm long×0.3 mm thick, with square pore opening of 200 μm and fiber diameter of 400 μm. Smooth muscle cell were seeded using a static seeding method: 500 μl of smooth muscle cell suspension (1×10⁵ cells/ml) was pipetted onto the upper surface of the construct and allowed to attach for 2 h at 37° C., before flooding with medium. After being seeded with cells, cell culture constructs were maintained in the well plates submerged in growth medium, and cultured at 37° C. in an incubator in a 90% humidified atmosphere of 5-10% carbon dioxide in air. Cell culture growth medium consisted of Dulbecco's Modified Eagle's Medium (DMEM) containing 5% (v/v) fetal bovine serum. In the case using a dynamic seeding, method, seeding was performed by immersing cell culture constructs in cell suspension within a spinner flasks stirred at 60 rpm, and contained at 37° C. in a humidified 5% CO₂ incubator. After seeding, cell culture constructs were placed into wells of tissue culture plate with medium for further culture at 37° C. in a humidified 5% CO₂ incubator. Culture medium was replaced regularly.

After cell culture was finished at certain time point, the cell culture constructs were taken out of the cell culture plate and underwent conventional assays. Cell culture constructs were disassembled in order to visualize, under a microscope, the cellular attachment and cellular activities on different layers or locations of the cell culture constructs.

In the case where the cells need to be recovered, the cells were trypsinized using Trypsin-EDTA solution (Sigma T4049). After detaching from cell culture constructs, cells were re-suspend in a small volume of fresh serum-containing medium to inactivate the trypsin. These cells then could be used for other purposes.

### 5.7 EXAMPLE 3

### Use of Cell Culture Construct for Cell Culture in a Bioreactor

The present invention also provides methods of using the cell culture construct for culturing living cells within a bioreactor. The cell culture construct used here was a disc shape (10 mm diameter discs with a thickness of 0.8 mm, porosity 80% and fiber diameter of 200 μm) and fit into the bioreactor.

Rat bone marrow stromal cells (MSCs) were statically seeded first onto the cell culture construct. 500 μl of MSC suspension with 250,000 rat MSCs was pipetted onto the upper surface of the cell culture construct, and allowed to attach for 2 hours at 37° C. before flooding with medium. After seeding with cells, these seeded cell culture constructs were maintained in a flow perfusion culture bioreactor. These cell seeded cell culture constructs were submerged in a complete osteo-differentiation medium, and cultured at 37° C. in a 90% humidified atmosphere of 5-10% carbon dioxide in air. The operation of the bioreactor system was driven by a peristaltic pump set at a rate of 1 ml/min. During the culture period, the culture medium was agitated to pass through the cell culture construct via the pores of the construct. Therefore, the cells were cultured under a dynamic shearing condition. The cells were cultured in the bioreactor for 4, 8, and 16 days, with a complete media exchange every 48 h.

At the end of the culture period, all cell culture construct constructs were rinsed with PBS and stored in 1.5 ml of distilled, deionized water at −20° C. until further analysis. Cell culture constructs were disassembled in order to visualize, under a microscope, the cellular attchment and cellular activities on different layers or locations of the cell culture constructs.

1. A three dimensional porous cell culture construct comprising struts and/or fibers joined in a rigid porous three dimensional designed pattern for cells to attach in cell culture medium.

2. A three dimensional porous cell culture construct comprising struts and/or fibers joined in a porous three dimensional designed pattern for cells to attach in cell culture medium, the construct having an average pore size of between 15 microns and 1000 microns, between 25 microns and 500 microns, or between 50 microns and 100 microns.

3. A three dimensional porous cell culture construct comprising struts and/or fibers joined in a porous three dimensional designed pattern for cells to attach in cell culture medium, the construct having a pore distribution of greater than about 50%, greater than about 80% or greater than about 95%.

4. The three dimensional porous cell culture construct of claim 1, wherein the struts and/or fibers are woven together or joined at angles to each other.

5. The three dimensional porous cell culture construct of claim 4, wherein the angle is selected from the group consisting of perpendicular, acute and obtuse.

6. The three dimensional porous cell culture construct of claim 1, wherein the struts and/or fibers comprise a polymer.

7. The three dimensional porous cell culture construct of claim 6, wherein the polymer is selected from the group consisting of polystyrene, polyethylene, polypropylene, polycarbonate, polyethylene terephthalate, polyamide, polyvinyl chloride and a combination thereof.

8. The three dimensional porous cell culture construct of claim 1, wherein the struts and/or fibers have a constant diameter.

9. The three dimensional porous cell culture construct of claim 1, wherein the struts and/or fibers have different diameters.

10. The three dimensional porous cell culture construct of claim 1, having cross sections selected from the group consisting of a circle, triangle, square, rectangle, star, irregular shape or a combination thereof.

11. The three dimensional porous cell culture construct of claim 1, wherein the struts and/or fibers are arranged from a base in a manner selected from the group consisting obliquely, horizontally, vertically and a combination thereof.

12. The three dimensional porous cell culture construct of claim 1, the pores have an average pore size of greater of between 15 microns and 1000 micros, between 25 microns and 500 microns, or between 50 microns and 100 microns.

13. The three dimensional porous cell culture construct of claim 1, wherein the pores have a constant size and/or dimension.

14. The three dimensional porous cell culture construct of claim 1, wherein the pores have a variable size and/or dimension.

15. The three dimensional porous cell culture construct of claim 1, wherein the pores on a plane horizontal to the base of the construct have a constant size and/or dimension.

16. The three dimensional porous cell culture construct of claim 1, wherein a pore on a plane horizontal to the base of the construct has a different size and/or dimension from a pore on another plane horizontal to the base of the construct.

17. The three dimensional porous cell culture construct of claim 1, wherein a pore on a plane horizontal to the base of the construct has the same or decreased size and/or dimension as a pore on an adjacent plane further from the base.

18. The three dimensional porous cell culture construct of claim 17, wherein a pore on at least one plane horizontal to the base of the construct has a decreased size and/or dimension as a pore on an adjacent plane further from the base.

19. The three dimensional porous cell culture construct of claim 1, wherein the pores on a plane horizontal to the base have a different size and/or dimension than the pores on the same plane.

20. The three dimensional porous cell culture construct of claim 1, further comprising a biomolecule impregnated in the construct.

21-56. (canceled)
